(19) United States
(12) Patent Application Publication
(10) Pub. No.: US 2020/0163654 A1
(43) Pub. Date: May 28, 2020

(54) METHOD AND APPARATUS TO CALIBRATE ULTRASOUND TRANSDUCERS

(71) Applicant: Butterfly Network, Inc., Guilford, CT (US)

(72) Inventors: Sarp Satir, San Francisco, CA (US); Jaime Scott Zahorian, Guilford, CT (US)

(73) Assignee: Butterfly Network, Inc., Guilford, CT (US)

(21) Appl. No.: 16/696,883

(22) Filed: Nov. 26, 2019

Related U.S. Application Data
(60) Provisional application No. 62/772,454, filed on Nov. 28, 2018.

(51) Int. Cl. A61B 8/00; A61B 8/08

(52) U.S. Cl. CPC A61B 8/58; A61B 8/5269; A61N 7/02; A61B 8/546; A61B 8/4494

(57) ABSTRACT

The disclosed embodiments relate to a capacitive micromachined transducers for ultrasound imaging having pressure calibrator to compensate for ultrasound image distortions caused by environmental pressure changes. In one embodiment, the disclosure relates to a method to calibrate a first ultrasound transducer of an array of ultrasound transducers for ambient pressure variation. The method includes the steps of detecting a real-time ambient pressure value; determining a pressure difference value between the detected ambient pressure value and a predetermined pressure value; and calibrating the first ultrasound transducers to compensate for the determined pressure difference.

### CROSS-REFERENCE TO RELATED APPLICATIONS

This application claims the benefit under 35 U.S.C. § 119(e) of U.S. Provisional Application Ser. No. 62/772,454, filed on Nov. 28, 2018 under Attorney Docket No. B1348.70104US00, and entitled “METHOD AND APPARATUS TO CALIBRATE ULTRASOUND TRANSDUCERS.” which is hereby incorporated herein by reference in its entirety.

### BACKGROUND

The present application relates to ultrasound devices having a pressure sensor calibrator. Specifically, the disclosed embodiments relate to micromachined ultrasonic transducers for ultrasound imaging or therapy having pressure calibrator to compensate for ultrasound image distortions caused by environmental pressure changes.

### RELATED ART

Ultrasound devices may be used to perform diagnostic imaging and/or treatment. Ultrasound imaging may be used to see internal soft tissue body structures. Ultrasound imaging may be used to find a source of a disease or to exclude any pathology. Ultrasound devices use sound waves with frequencies which are higher than those audible to humans. Ultrasonic images are made by sending pulses of ultrasound into tissue using a probe. The sound waves are reflected off the tissue, with different tissues reflecting varying degrees of sound. These reflected sound waves may be recorded and displayed as an image to the operator. The strength (amplitude) of the sound signal and the time it takes for the wave to travel through the body provide information used to produce an image.

Many different types of images can be formed using ultrasound devices. The images can be real-time images. For example, images can be generated that show two-dimensional cross-sections of tissue, blood flow, motion of tissue over time, the location of blood, the presence of specific molecules, the stiffness of tissue, or the anatomy of a three-dimensional region.

### SUMMARY

In an exemplar embodiment, the disclosure relates to a method to calibrate a first ultrasound transducer of an array of ultrasound transducers for ambient pressure variation. An exemplary method includes the steps of: detecting a real-time ambient pressure; determining a pressure difference between the detected ambient pressure and a predetermined pressure; calibrating the first ultrasound transducers to compensate for the determined pressure difference.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates an example of an ultrasound probe configured to perform a sequence of acquisitions according to one embodiment of the disclosure.

FIG. 2 illustrates an ultrasound probe architecture according to some embodiments of the disclosure.

FIG. 3 illustrates an ultrasound probe for ultrasound imaging according to one embodiment of the disclosure.

FIG. 4A schematically illustrates top view of an exemplary ultrasound probe head.

FIG. 4B schematically illustrates an exemplary ultrasonic transducer.

FIG. 5 shows an exemplary flow-diagram for calibrating one or more transducers according to one embodiment of the disclosure.

FIG. 6 schematically shows a calibration apparatus according to one embodiment of the disclosure.

### DETAILED DESCRIPTION

Various aspects and embodiments of the application will be described with reference to the following figures. It should be appreciated that the figures are not necessarily drawn to scale. Items appearing in multiple figures are indicated by the same reference number in all the figures in which they appear.

Ultrasound devices may be used to perform diagnostic imaging and/or treatment, using sound waves with frequencies that are higher than those audible to humans. Ultrasound imaging may be used to see internal soft tissue body structures, for example to find a source of disease or to exclude any pathology. When pulses of ultrasound are transmitted into tissue (e.g., by using a probe), sound waves are reflected off the tissue, with different tissues reflecting varying degrees of sound. These reflected sound waves may then be recorded and displayed as an ultrasound image to the operator. The strength (amplitude) of the sound signal and the time it takes for the wave to travel through the body provide information used to produce the ultrasound image. Many different types of images can be formed using ultrasound devices, including real-time images. For example, images can be generated that show two-dimensional cross-sections of tissue, blood flow, motion of tissue over time, the location of blood, the presence of specific molecules, the stiffness of tissue, or the anatomy of a three-dimensional region.

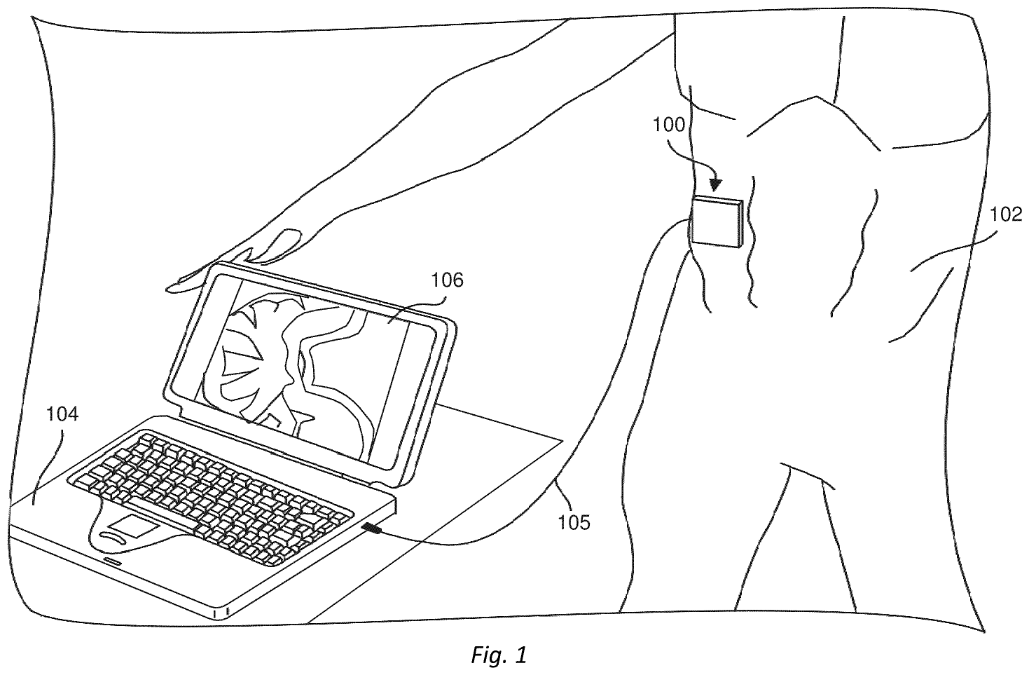

FIG. 1 illustrates an example of an ultrasound probe configured to perform a sequence of acquisitions in accordance with some embodiments of the technology described herein. Specifically, FIG. 1 illustrates an example of an ultrasound probe 100 configured to perform imaging in a desired imaging mode in response to receiving an indication to begin imaging in the desired imaging mode. The ultrasound probe 100 is shown for purposes of illustration as being used to investigate a subject 102. As illustrated in the embodiment of FIG. 1, ultrasound probe is coupled to host device 104 via a connection 105. The host device 104 may provide an initiation command to the ultrasound probe 100 to begin imaging in a desired imaging mode (e.g., in any of the imaging modes described herein), and in response to receiving the initiation command, ultrasound probe 100 may commence imaging in the desired imaging mode using control data stored in the ultrasound probe's memory (not shown in FIG. 1).

Host device 104 may be any suitable computing device and may be a portable computing device (e.g., a laptop, smartphone, a tablet, a personal digital assistant, a computing device affixed to portable medical equipment, etc.) or a fixed computing device (e.g., a desktop computer, a rack mount computer, a computing device affixed to other fixed medical equipment, etc.). In the illustrated embodiment, host device 104 includes display screen 106 on which ultrasound images may be displayed in real time, substantially in real time as imaging is performed (e.g., within a threshold number of frames such as within one, five or ten frames, within a threshold amount of time such as within one, five, or ten seconds, etc.), or after imaging is performed, though in other embodiments host device 104 may not have a display screen.

In the embodiment of FIG. 1, connection 105 is a wired connection, but may be a wireless connection (e.g., a Bluetooth® connection or near field communication (NFC)), as aspects of the technology described herein are not limited in this respect. The connection 105 may be a digital connection, for example being of a type commonly used with commercial digital electronics, such as a universal serial bus (USB) cable, Thunderbolt, or FireWire, Connection 105 may connect to the serial output port 314 and clock input port 316 of the ultrasound probe 100.

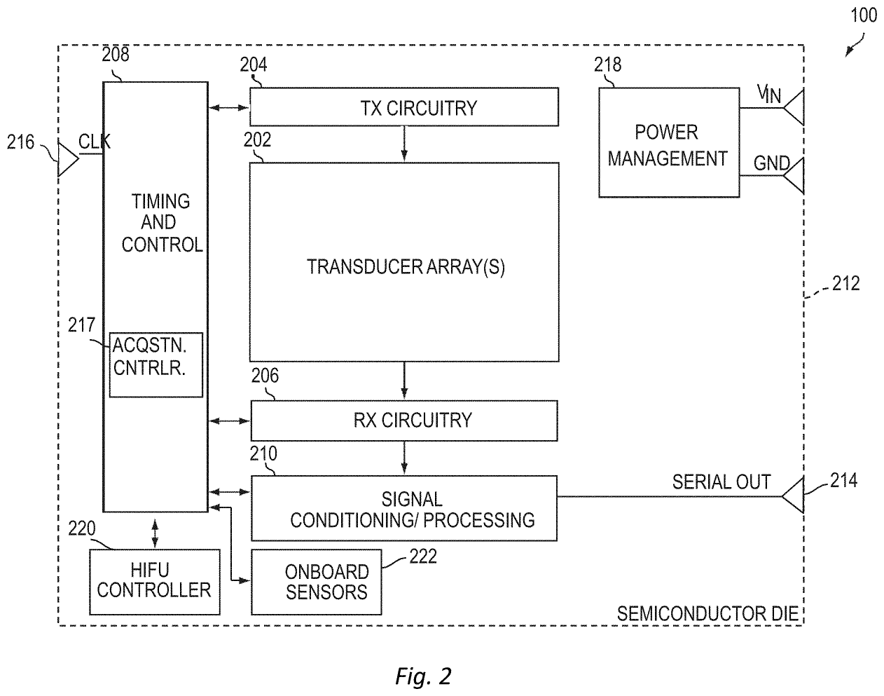

FIG. 2 illustrates components of ultrasound probe 100, in accordance with some embodiments of the technology described herein. The ultrasound probe 100 includes one or more transducer arrangements (e.g., arrays) 202 of ultrasonic transducers, transmit (TX) circuitry 204, receive (RX) circuitry 206, timing and control circuitry 208, signal conditioning/processing circuitry 210, and/or a power management circuit 218 receiving ground (GND) and voltage reference (VIN) signals. Transmit circuitry 204 may assume different embodiments without departing form the disclosed principles.

The ultrasound probe 100 may include acquisition controller 217, which may be implemented as a processor, for controlling other circuitry of the ultrasound probe to perform a sequence of acquisitions governed by control data stored on the ultrasound probe. Acquisition controller 217 may be part of the timing and control circuitry 208, or may be separate in other embodiments. In general, the timing and control circuitry 308 may include suitable circuitry for controlling operation of the transmit circuitry 204, receive circuitry 206, onboard sensors 222, and/or any other suitable components of ultrasound probe 100. Optionally, a high intensity focused ultrasound (HIFU) controller 220 may be included if the ultrasound probe 100 is to be used to provide HIFU.

The ultrasound probe 100 may include one or more onboard sensors 222, which may sense data about the probe and/or its environment. Sensors 222 are “onboard” in the sense that they may be integrated with ultrasound probe 100, which may be done in any suitable way. For example, onboard sensors 222 may be discrete components on ultrasound probe 100, may be integrated with the ultrasound transducers on the same substrate, etc. Sensors 222 may include one or more non-acoustic sensors of any suitable type and, for example, may include one or more accelerometers, gyroscopes, or other sensors indicating movement of the probe, one or more temperature sensors indicating the temperature of the probe (e.g., the temperature of the probe's circuitry), one or more sensors indicating an amount of power used by the probe, one or more pressure sensors, and/or any other suitable type(s) of sensors.

The onboard sensors 222 may obtain data about the probe and/or its environment when the probe is performing imaging, and the probe (e.g., acquisition controller 217) may adapt the way in which it performs the imaging, processes data acquired during imaging, and/or initiates imaging based at least in part on the data acquired by onboard sensors 222. For example, onboard sensors 222 may obtain data indicating that the probe has moved (e.g., a handheld probe may be moved inadvertently due to movement of the user's hand) and the probe may use the obtained data to adjust the way in which it performs imaging to account for the motion (e.g., by using beam steering or other techniques to continue imaging the same portion of the subject as was being imaged prior to the motion or by suitably post-processing the acquired data to account for the motion). As another example, onboard sensors 222 may obtain data indicating that the temperature of at least one component of the probe (e.g., the probe's circuitry) has exceeded a desired threshold and the probe may adjust the way in which it performs imaging to reduce the probe's temperature (e.g., by reducing the power of the transmitted pulses, by reducing the frequency at which the probe emits pulses, by performing less processing of the acquired data, etc.). As yet another example, onboard sensors 222 may obtain data indicating that the power used by the probe has exceeded a desired threshold and the probe may adjust the way in which it performs imaging to reduce the amount of power utilized by the probe (e.g., by reducing the power of the transmitted pulses, by reducing the frequency at which the probe emits pulses, by reducing the number of ultrasonic elements used to transmit and/or receive data, etc.) relative to the amount of power that would have been used by the probe if it continued to perform imaging without adjustment. It should be appreciated that the onboard sensors 222 may also collect data when the probe is not performing imaging and the collected data may be used to control the manner in which imaging is subsequently performed and/or the way data acquired as a result of imaging is processed.

In some embodiments, ultrasound probe 100 may receive an indication to perform an acquisition task (e.g., from host device 104), receive non-acoustic data obtained by one or more of the onboard sensors 222, and control, based on the non-acoustic data and control data for the acquisition task stored on the ultrasound probe 100, the ultrasound probe 100 to obtain acoustic data for the acquisition task. This may be done in any suitable way. For example, in some embodiments, the control data may comprise multiple parameters governing performance of the acquisition task and one or more of the multiple parameters may be selected, based on the non-acoustic data, and used for controlling the ultrasound probe 100 to obtain acoustic data for the acquisition task. In some embodiments, the control data may comprise multiple parameters governing performance of the acquisition task and the value(s) of one or more of the multiple parameters may be adjusted, based on the non-acoustic data, to obtain parameter values to be used in controlling the ultrasound probe 100 to obtain acoustic data for the acquisition task.

Any suitable component(s) of ultrasound probe 100 may be controlled based, at least in part, on the non-acoustic data including, but not limited to, ultrasonic transducer arrangements 202, transmit circuitry 204, and receive circuitry 206.

In the embodiment shown in FIG. 2, all of the illustrated components are formed on a single semiconductor die (or substrate or chip) 212, and thus the illustrated embodiment is an example of an ultrasound on a chip device. However, not all embodiments are limited in this respect. In addition, although the illustrated example shows both TX circuitry 204 and RX circuitry 206, in alternative embodiments only TX circuitry or only RX circuitry may be employed. For example, such embodiments may be employed in a circumstance in which the ultrasound probe is operated as a transmission-only device to transmit acoustic signals or a reception-only device used to receive acoustic signals that have been transmitted through or reflected by a subject being ultrasonically imaged, respectively.

The ultrasound probe 100 further includes a serial output port 214 to output data serially to a host (e.g., serial output port 214 may be used to output data to a host device). The ultrasound probe 100 may also include a clock input port 216 to receive a clock signal (e.g., from a host device such as device 104) and provide the received clock signal CLK to the timing and control circuit 208.

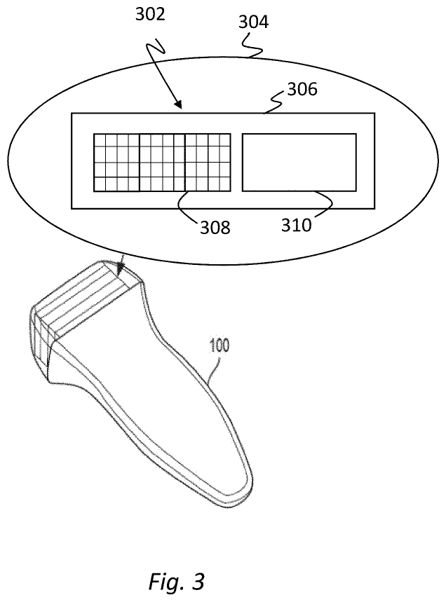

FIG. 3 illustrates an ultrasound probe with a simplified schematic of an ultrasound-on-a-chip device that may be used in the probe. Probe 300 may be a handheld probe configured to plug into a computer, smartphone, tablet, or other external device, or to communicate wirelessly with such a device. The probe 300 may include an ultrasound-on-a-chip device 302, shown in the call-out view 304. The ultrasound-on-a-chip device 302 may include a substrate 306 with integrated ultrasonic transducers 308 and circuitry 310. For ease of illustration, the schematic view of FIG. 3 shows the ultrasonic transducers 308 and circuitry 310 as simplified blocks in a side-by-side configuration. As is illustrated in subsequent figures, and described further below, the physical implementation may have the ultrasonic transducers and circuitry in a stacked configuration. Substrate 306 may be a semiconductor substrate, such as a silicon or silicon-on-insulator (SOI) substrate, and in some embodiments is a complementary metal oxide semiconductor (CMOS) substrate. Ultrasonic transducers 308 may be capacitive micromachined ultrasonic transducers (CMUTs) and the circuitry 310 may be integrated circuitry, such as silicon circuitry.

In the simplified representation of call-out view 304, the ultrasonic transducers 308 and circuitry 310 are schematically depicted as being side-by-side for purposes of illustration. In practice, such a side-by-side configuration is physically possible on a substrate, but so are alternatives. In certain embodiments, substrate 306 is formed by bonding an engineered substrate having ultrasonic transducers with a substrate having an IC (or other electrical substrates, such as silicon interposers or other types of interposers, or printed circuit boards), such that the ultrasonic transducers and IC may be in a stacked configuration. In another embodiment, substrate 306 may comprise one or more sensors (e.g., pressure sensor) to detect ambient information. Such ambient information may include, for example, ambient temperature and pressure.

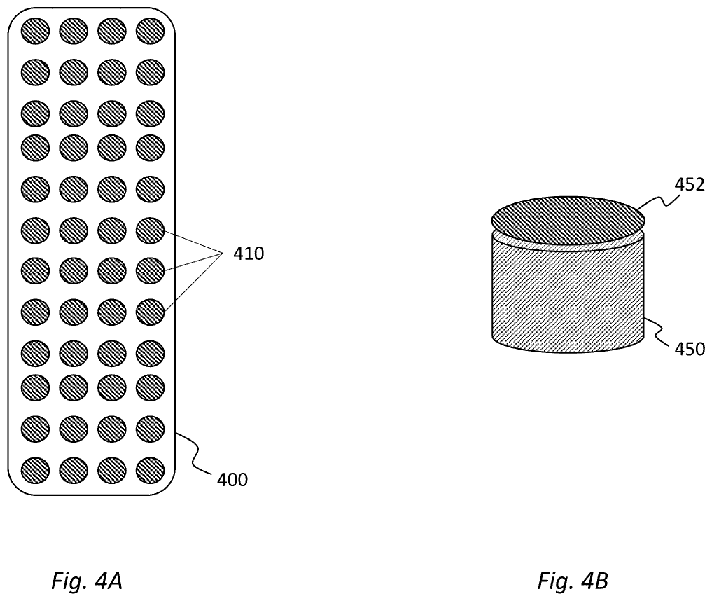

FIG. 4A schematically illustrates top view of an exemplary ultrasound probe head. Specifically, FIG. 4A shows the tops of ultrasound transducers 410 arranged on substrate 400. Each instance of 410 may depict a membrane associated with a respective ultrasound cavity. Substrate 400 may comprise additional components, such as integrated circuitry (not shown) and sensors (not shown).

FIG. 4B schematically illustrates an exemplary ultrasonic transducer. In FIG. 4B, transducer 450 may be formed in a bulk silicon substrate (e.g., substrate 400). Transducer 450 may be formed as a cylindrical cavity in a bulk silicon substrate (not shown). Transducer 450 may also include one or more electrodes (not shown). The electrode (not shown) may form a capacitive coupling with membrane 452. Membrane 452 is configured to vibrate in order to generate and transmit ultrasound waves (i.e., transmit mode). Membrane 452 is also configured to detect and receive incoming (or rebound) ultrasound wave forms. In an exemplary embodiment, each transducer 450 comprises a membrane 452.

Positioning of membrane 452 over the cavity of a transducer 450 is critical in that anomalies or variations in the membrane's position can have significant implications for the received signal. For example, if the membrane is not properly disposed over the cavity of the transducer, the received signal may distort the ultrasound image. In another example, if the membrane subsides or bulges (downward or upward) the ultrasound image may be distorted.

One such distortion is caused by external (e.g., ambient) pressure. When the external pressure is higher than the pressure inside the transducer cavity, the membrane (or portions thereof) may concave towards the transducer cavity. Alternatively, when the external pressure is lower than the pressure inside the transducer cavity, the membrane may bulge outwardly or form a convex surface relative to the substrate.

To address this and other deficiencies, an embodiment of the disclosure provides a method, system and apparatus to identify a relative pressure (or relative vacuum) and calibrate the ultrasound signals from the membrane accordingly. In one embodiment, an external sensor is used to detect ambient pressure and determine whether the ambient pressure is significant enough to calibrate the output signal of an affected transducer membrane.

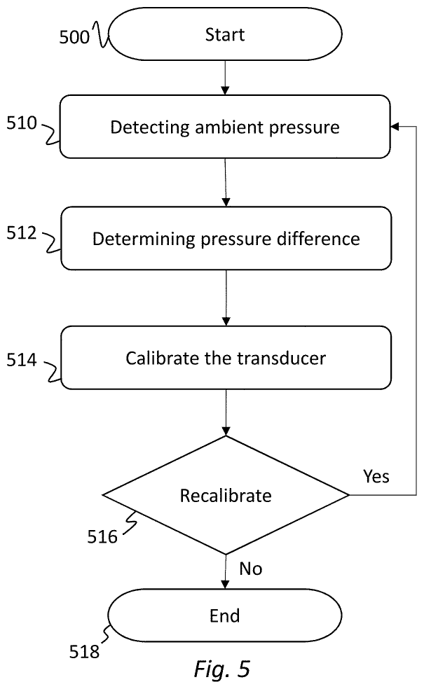

FIG. 5 shows an exemplary flow-diagram for calibrating one or more transducers according to one embodiment of the disclosure. The flow-diagram of FIG. 5 may be implemented by processor circuitry. Memory circuitry may store instructions for executing a method as illustrated in the flow-diagram of FIG. 5.

The process of FIG. 5 starts at step 500. In one embodiment, step 500 is triggered by an external event. For example, step 500 may be triggered when the probe (e.g., probe 100, FIG. 1) is turned on. In another embodiment, step 500 is triggered during certain time intervals, for example, after every 3 minutes of probe operation. In still another embodiment, step 500 is triggered upon a probe operator's request.

At step 510, external pressure is detected. The external pressure may be detected using one or more sensors. The detected pressure may denote real-time ambient pressure in the environment where the probe is operating. For example, if the probe is used inside a pressure chamber, the detected ambient pressure would denote the pressure inside the chamber. The sensors may be pressure sensors to measure ambient pressure at the environment where the ultrasound probe is used. The one or more sensors may be integrated in the probe. In one embodiment, the sensors are integrated in the circuitry that houses the ultrasound chip. For example, the one or more pressure sensors may be integrated with circuitry 310 of FIG. 3.

In certain embodiments of the disclosure, ambient pressure is estimated as a function of the capacitive baseline measurements. The capacitive baseline measurement can be used and correlate to the detected ambient pressure. In an exemplary application, a baseline noise level output for a capacitive transducer can be measured. The baseline may define deflection in the transducer membrane in the absence of any input signal to the transducer or to the membrane. In some embodiments, the baseline deflection can be measured by the transducer output noise. Thus, any initial deflection (i.e., noise) in the membrane may be used to correlate noise to ambient pressure. In another embodiment, the deflection can define the pressure change from the base line or from a previous deflection level.

In an exemplary embodiment, the baseline noise level of one or more transducers is measured and used as an indication of an initial deflection in the membrane. A lookup table correlating membrane deflection to ambient pressure may be apriori stored at an accessible database. The detected initial deflection (i.e., baseline noise) can be correlated to an ambient pressure based on the information considered in the lookup table.

At step 512, the pressure difference value is determined. The pressure difference value may be determined as a difference between the value detected in step 510 (e.g., pressure detected by a sensor or pressure detected based on baseline noise) and a predefined pressure. The predefined pressure may be an arbitrary pressure value. For example, the predetermined pressure value may be atmospheric pressure at sea level.

At step 514, the transducer is calibrated to compensate for the determined pressure difference. The difference between real-time and the predetermined pressure can be used to calibrate any reading from the transducer. In other words, the difference between a real-time deflection in the transducer membrane and the expected deflection in the transducer membrane can be used to calibrate the transducer. The calibration value may be used to compensate an ultrasound image obtained from the transducer.

At step 516, recalibration determination is made. If additional calibration is deemed appropriate, the process reverts back to step 510. If additional calibration is not required, the process ends at step 518.

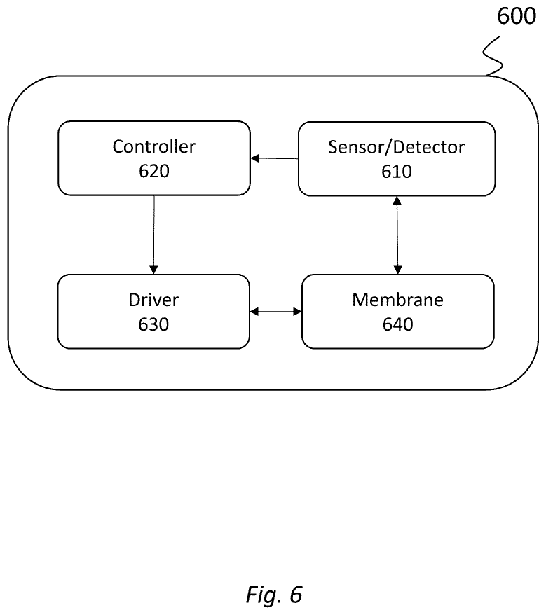

FIG. 6 schematically shows a calibration apparatus according to one embodiment of the disclosure. Apparatus 600 of FIG. 6 includes sensor (interchangeably, detector) 610, controller 620, driver 630 and membrane 640. Apparatus 600 may be implemented in hardware or a combination of hardware and software (e.g., firmware). Apparatus 600 may be incorporated into an ultrasound probe. In one embodiment, apparatus 600 is integrated with a circuitry configured to operate the ultrasound probe. The ultrasound probe can be handheld, portable, ultrasound probe as disclosed herein. In certain embodiments, apparatus 600 may be implemented as an independent chipset. In other embodiments, apparatus 600 may be integrated with an existing semi-conductor die (e.g., semiconductor die 100 of FIG. 2).

Sensor 610 may define a conventional pressure sensor or any device that can detect a pressure change. In one embodiment, sensor 610 comprises a transducer configured to detect pressure change.

In certain embodiments, sensor 610 comprises a detector configured to communicate with membrane 640 and detect a deflection in the membrane. The membrane deflection may be determined as any deflection (e.g., sagging or bowing) in the membrane as compared to a predefined level. For example, a membrane may be deflected inwardly (i.e., sagging) due to increased ambient pressure). Detector 610 may detect, identify and register the deflection as compared with the predefined, normal, level. Membrane 640 can be associated with a transducer cavity (not shown). Sensor 610 may comprise a sensor and/or detector (e.g., hardware and software) in communication with membrane 640 and configured to detect any deflection or inflation of membrane 640; the results of which may be correlated to an external pressure estimate.

Sensor/Detector 610 may communicate the detected deflection to Controller 620. Controller 620 may comprise hardware, software or a combination of hardware and software (e.g., firmware). Controller 620 may comprise one or more processors circuitry (not shown) in communication with one or more memory circuitry (not shown) to receive input signal from Sensor/Detector 610. Controller 620 may comprise, or communicate with, a memory (e.g., database or a table) circuitry. The memory circuitry may include a database correlating membrane deflection with ambient pressure. For example, the database may include a table correlating each additional pressure points with a corresponding deflection (or inflation) of the membrane as measured from a baseline. Controller 620 may compare information from the input signal to determine whether sensor calibration is required.

In one embodiment, when controller 620 determines that sensor calibration is required, the controller signals driver 630 to adjust bias signal to membrane 640. The adjusted bias signals to the sensor can cause membrane 640 to move to a baseline position. Thereafter, the membrane movements will be calibrated by the additional bias to compensate for the detected pressure difference. Driver 630 may comprise hardware and/or software required to drive membrane 640. In certain embodiments, controller 620 may engage driver 630 to thereby cause membrane 640 to deflect in a predetermined amount consistent with available information and the estimated ambient pressure. By way of example, controller 620 may determine that membrane 640 is deflected in response to external pressure by a determined amount. Controller may then determine (e.g., using a look up table stored in the memory) that the membrane deflection is due to a known amount of external pressure. Controller 620 may then direct driver 630 to deflect membrane 640 by an amount required to bring membrane 640 to a substantially flat (or predetermined baseline) level. In one example, driver 630 may comprise a voltage regulator to bias membrane 640 to a base level. Thereafter, any additional pressure sensed by membrane 640 can be used to measure ultrasound wave without ambient pressure distortion.

In FIG. 6, membrane 640 may represent a membrane from a single transducer. Alternatively, membrane 640 in FIG. 6 may represent a plurality (e.g., a group) of membranes of a CMUT chip. In an exemplary embodiment, the signal may be an averaged value from a group of membranes 640 to represent an average deflection in a group of membranes. For example, a group of membranes in the array of membranes may be selected and an average deflection value may be assessed on the group of membranes.

In an alternative embodiment, when sensor calibration is required, the controller may use the offset information to calibrate any signal coming from membrane 640. Here, an external calibration is not made to the transducer's membrane; instead, the signal coming from the transducer is adjusted to account for any pressure calibration offset.

The disclosed embodiments may be used with a CMUT-type transducers. The disclosed embodiments may be equally applied to piezoelectric-type transducers. For example, the disclosed embodiments may be applied to one or more transducers having piezoelectric membranes. Using the disclosed principles, membrane deflection due to external factors (e.g., pressure or temperature) can be determined and the transducer membrane(s) may be calibrated accordingly.

The following non-limiting example are provided to illustrate different embodiments consistent with the disclosed principles.

Example 1 is directed to an ultrasound device, comprising an ultrasound-on-a-chip device comprising an array of ultrasonic transducers; and a pressure sensor configured to detect pressure applied to an ultrasonic transducer of the array.

Example 2 is directed to the ultrasound device of example 1, wherein the pressure sensor is integrated with the ultrasound on-a-chip device.

Example 3 is directed to a method to calibrate a first ultrasound transducer of an array of ultrasound transducers for ambient pressure variation, the method comprising: detecting a real-time ambient pressure value; determining a pressure difference value between the detected ambient pressure value and a predetermined pressure value; and calibrating the first ultrasound transducers to compensate for the determined pressure difference.

Example 4 is directed to the method of example 3, wherein detecting a real-time ambient pressure further comprises measuring the ambient pressure with a pressure sensor.

Example 5 is directed to the method of example 3, wherein detecting a real-time ambient pressure further comprises measuring a noise level output of the first ultrasound transducer and correlating the noise level output to ambient pressure and wherein the noise level output defines the noise output of the first ultrasound transducer absent an input signal to the first transducer.

Example 6 is directed to the method of example 3, wherein detecting a real-time ambient pressure further comprises measuring an average noise level output per transducer for the array of ultrasonic transducers and correlating the average noise level output to ambient pressure and wherein the average noise level output defines the averaged noise output of the array of ultrasound transducers absent an input signal to the array.

Example 7 is directed to the method of example 5, wherein measuring a noise level output of at least one of the ultrasonic transducers further comprises detecting background noise of a first transducer by measuring the first transducer's noise output in the absence of an input signal to the first transducer.

Example 8 is directed to the method of example 3, wherein determining a pressure difference further comprises comparing the real-time ambient pressure value with a predefined pressure value.

Example 9 is directed to the method of example 3, wherein the step of calibrating the first ultrasound transducers further comprises biasing the first ultrasound transducer to a first bias value to cause a predetermined deflection in a membrane of the first ultrasound transducer.

Example 10 is directed to the method of example 3, wherein the step of calibrating the first ultrasound transducers further comprises adjusting the image quality of a received signal from the first ultrasound transducer to compensate for the pressure difference.

Example 11 is directed to the method of example 10, further comprising dynamically changing an image parameter to compensate for the pressure difference.

Example 12 is directed to an ultrasound transducer device, comprising: an array of ultrasound transducers including a first transducer in the array, wherein the first transducer further comprises a first capacitive micromachined transducer (CMUT) with a first membrane; a detector in communication with the first transducer, the detector configured to detect a deflection value in the first membrane; and a controller in communication with the CMUT, the controller configured to receive the deflection value from the detector, determine a pressure difference value between the detected ambient pressure and a predetermined pressure and compensate for the determined pressure difference value.

Example 13 is directed to the ultrasound device of example 12, wherein the detector is an ambient pressure sensor.

Example 14 is directed to the ultrasound device of example 12, wherein the detector is configured to measure a noise level output of the first ultrasound transducer and correlate the noise level output to ambient pressure and wherein the noise level output defines the noise output of the first ultrasound transducer absent an input signal to the first transducer.

Example 15 is directed to the ultrasound device of example 12, wherein the detector is configured to correlate the first membrane's deflection in the absence of an input signal as a measure of ambient pressure.

Example 16 is directed to the ultrasound device of example 12, wherein the detector is configured to detect an average noise level output per transducer for the array of ultrasonic transducers and to correlate the average noise level output from the array of ultrasound transducers.

Example 17 is directed to the ultrasound device of example 12, wherein the detector is configured to measure a noise level output of the first transducer by measuring the first transducer's noise output in the absence of an input signal.

Example 18 is directed to the ultrasound device of example 12, wherein the controller compensates for the determined pressure difference value by biasing the first membrane.

Example 19 is directed to the ultrasound device of example 12, wherein the controller compensates for the determined pressure difference value by biasing a respective membrane associated with each transducer in the array.

Example 20 is directed to the ultrasound device of example 12, wherein the controller compensates for the determined pressure difference by adjusting an image quality of a received signal from the array of transducers.

Example 21 is directed to the ultrasound device of example 12, wherein the array of ultrasound transducers, the detector and the controller are integrated in a solid-state device.

Example 22 is directed to the ultrasound device of example 12, wherein the array of ultrasound transducers, the detector and the controller are integrated to form a chipset.

While the principles of the disclosure have been illustrated in relation to the exemplary embodiments shown herein, the principles of the disclosure are not limited thereto and include any modification, variation or permutation thereof.

What is claimed is:

1. An ultrasound device, comprising:
an ultrasound-on-a-chip device comprising an array of ultrasonic transducers; and
a pressure sensor configured to detect pressure applied to an ultrasonic transducer of the array.

2. The ultrasound device of claim 1, wherein the pressure sensor is integrated with the ultrasound on-a-chip device.

3. A method to calibrate a first ultrasound transducer of an array of ultrasound transducers for ambient pressure variation, the method comprising:
detecting a real-time ambient pressure value;
determining a pressure difference value between the detected ambient pressure value and a predetermined pressure value; and
calibrating the first ultrasound transducer to compensate for the determined pressure difference.

4. The method of claim 1, wherein detecting a real-time ambient pressure further comprises measuring the ambient pressure with a pressure sensor.

5. The method of claim 3, wherein detecting a real-time ambient pressure further comprises measuring a noise level output of the first ultrasound transducer and correlating the noise level output to ambient pressure and wherein the noise level output defines the noise output of the first ultrasound transducer absent an input signal to the first transducer.

6. The method of claim 3, wherein detecting a real-time ambient pressure further comprises measuring an average noise level output per transducer for the array of ultrasonic transducers and correlating the average noise level output to ambient pressure and wherein the average noise level output defines the averaged noise output of the array of ultrasound transducers absent an input signal to the array.

7. The method of claim 5, wherein measuring a noise level output of at least one of the ultrasonic transducers further comprises detecting background noise of a first transducer by measuring the first transducer's noise output in the absence of an input signal to the first transducer.

8. The method of claim 3, wherein determining a pressure difference further comprises comparing the real-time ambient pressure value with a predefined pressure value.

9. The method of claim 3, wherein the step of calibrating the first ultrasound transducer further comprises biasing the first ultrasound transducer to a first bias value to cause a predetermined deflection in a membrane of the first ultrasound transducer.

10. The method of claim 3, wherein the step of calibrating the first ultrasound transducers further comprises adjusting the image quality of a received signal from the first ultrasound transducer to compensate for the pressure difference.

11. The method of claim 10, further comprising dynamically changing an image parameter to compensate for the pressure difference.

12. An ultrasound transducer device, comprising:
an array of ultrasound transducers including a first transducer in the array, wherein the first transducer further comprises a first capacitive micromachined transducer (CMUT) with a first membrane;
a detector in communication with the first transducer, the detector configured to detect a deflection value in the first membrane; and
a controller in communication with the CMUT, the controller configured to receive the deflection value from the detector, determine a pressure difference value between the detected ambient pressure and a predetermined pressure and compensate for the determined pressure difference value.

13. The ultrasound device of claim 12, wherein the detector is an ambient pressure sensor.

14. The ultrasound device of claim 12, wherein the detector is configured to measure a noise level output of the first ultrasound transducer and correlate the noise level output to ambient pressure and wherein the noise level output defines the noise output of the first ultrasound transducer absent an input signal to the first transducer.

15. The ultrasound device of claim 12, wherein the detector is configured to correlate the first membrane's deflection in the absence of an input signal as a measure of ambient pressure.

16. The ultrasound device of claim 12, wherein the detector is configured to detect an average noise level output per transducer for the array of ultrasonic transducers and to correlate the average noise level output from the array of ultrasound transducers.

17. The ultrasound device of claim 12, wherein the detector is configured to measure a noise level output of the first transducer by measuring the first transducer's noise output in the absence of an input signal.

18. The ultrasound device of claim 12, wherein the controller compensates for the determined pressure difference value by biasing the first membrane.

19. The ultrasound device of claim 12, wherein the controller compensates for the determined pressure difference value by biasing a respective membrane associated with each transducer in the array.

20. The ultrasound device of claim 12, wherein the controller compensates for the determined pressure difference by adjusting an image quality of a received signal from the array of transducers.

21. The ultrasound device of claim 12, wherein the array of ultrasound transducers, the detector and the controller are integrated in a solid-state device.

22. The ultrasound device of claim 12, wherein the array of ultrasound transducers, the detector and the controller are integrated to form a chipset.
